(19) United States
(12) Patent Application Publication
(10) Pub. No.: US 2017/0258404 A1
(43) Pub. Date: Sep. 14, 2017

(54) Coated Biosensor and Method for Preserving Biosensor During Implantation into the Brain or Other Tissues

(71) Applicant: DIAGNOSTIC BIOCHIPS, INC., Glen Burnie, MD (US)

(72) Inventors: Emma Bigelow, Baltimore, MD (US); Brian Jamieson, Severna Park, MD (US)

(73) Assignee: DIAGNOSTIC BIOCHIPS, INC., Glen Burnie, MD (US)

(21) Appl. No.: 15/529,625

(22) PCT Filed: Nov. 25, 2015
(86) PCT No.: PCT/US15/62630

§ 371 Date: May 25, 2017

Related U.S. Application Data
(60) Provisional application No. 62/084,185, filed on Nov. 25, 2014.

(51) Int. Cl. A61B 5/00; A61B 5/1486

(52) U.S. Cl. CPC A61B 5/6868; A61B 5/14865; A61B 2562/028; A61B 2562/063; A61B 2562/12

(57) ABSTRACT

Provided herein is a coated biosensor and a method of preserving a coated biosensor to protect it during implantation into the brain or other tissues by coating the biosensor with a protective coating.

### BACKGROUND

For chemical sensors in the brain, immune response and biofouling by blood during initial surgery presents a significant obstacle to in vivo sensing. If sensors could be delivered directly to healthy brain tissue surrounded by only cerebral spinal fluid, much less sensor biofouling would occur. Therefore, some protective technique is likely required in order to eventually have the most intact and responsive sensor possible in the brain.

Enzyme sensors used in the body regularly have a permanent coating, which is required to maintain the specificity of the sensor. These coatings result in poor temporal resolution of the sensors as diffusion of molecules to be sensed through the coating becomes a limiting factor. The permanent coatings used on enzyme sensors are thick and without any spatial resolution. Additionally, the potential immunogenicity of enzymes in the body precludes the use of a temporary coating on those sensors.

### SUMMARY

The present application provides a method for protecting a biosensor during implantation, comprising providing the sensor with a temporary coating. This coating will comprise one or more layers, each of which may comprise one or more the polyethylene glycol (PEG), carboxymethylcellulose, other hydrogels, silk protein, or chitosan, or the like. Such coatings will temporarily (minutes to days) protect aptamer, antibody, or enzyme based sensors during implantation and subsequent settling of brain tissue and immune response.

The use of the described temporary coating to protect a sensor for implantation may be assumed to be somewhat exclusive to aptamer-based biosensors, where immunogenicity is not an issue. As aptamer biosensors in vivo are a novel approach by DBC, methods around prolonging aptamer biosensor in vivo lifespan are similarly novel. Using photolithography or other methods of placing coatings over specific sensors on a microfabricated sensor is novel and may be required to achieve high precision of which sensors are exposed when.

With a temporary protective coating, biofouling substances such as red blood cells, clotting factors, and inflammatory cytokines stick to the outside coating surface and do not attach to the underlying sensor. Once the protective coating begins to dissolve or melt in physiological ionic solutions (CSF) or temperature, the biofouling substances are removed with the coating molecules (which are typically large molecules), thus leaving the biosensing layer relatively free of fouling substances. The use of the temporary protective coating(s) described herein This invention could either fully enable in vivo sensing, or just improve the quality of the sensor once it is in place, thereby improving the SNR, limit of detection, and dynamic range.

The temporary coatings described herein may also be used on biosensors for subcutaneous or intraperitoneal implantation for improved sensor preservation during placement.

This method will allow for improved sensitivity and specificity of a biosensor by preserving the number of biosensing elements available for binding after placement in the brain or other tissue. As a result, biosensors will last longer, have higher signal-to-noise ratios, and correspondingly improved limits of detection of dynamic ranges.

### BRIEF DESCRIPTION OF THE DRAWINGS

The elements in the drawings provided herein are not to scale.

FIG. 1A shows a schematic of an array 10 covered with a coating 20 which covers biosensing elements 30.

FIG. 1B shows a schematic of an array 10 where the coating 20 is applied in a manner such that the thickness of the coating 20 is greater at one end of the array 10 than at the other end of the array 10. A single variety of biosensing elements 30 is disposed on the array 10.

FIG. 1C shows a schematic of an array 10 where the coating 20 is applied in a manner such that the thickness of the coating 20 is greater at one end of the array 10 than at the other end of the array 10. Multiple varieties of biosensing elements 30, 31, 32, 33, 34 are disposed on the array 10.

FIG. 1D shows a schematic of an array 10 where the thickness of the coating 20 varies over the surface of the array because of the underlying topography of the array 10.

FIGS. 1E and 1F show a schematic of an array 10, which is covered by a coating 20. The array includes projections or pillars 11. Biosensing elements 30 may be on and/or between the pillars 11. FIG. 1F shows an embodiment in which the biosensing elements 31 on the pillars differ from the biosensing elements 32 which are between the pillars.

FIGS. 2A and 2B show a schematic of an array 10, which is covered by multiple coatings 20, 21, 22, 23, 24. In FIG. 2A all of the biosensing elements 30 are the same, while in FIG. 2B, each different coating covers a different biosensing element 30, 31, 32, 33, 34.

### DETAILED DESCRIPTION

The method described above for the coating of a biosensor before implantation requires the following components:

A functionalized biosensor (possible biosensing elements include aptamers, enzymes, antibodies, and novel biosensing molecules) is prepared on an electrode substrate (such as a microwire or microfabricated sensor). Suitable biosensing elements, and methods of making such elements, are well known in the art. Suitable electrode substrates are also well known in the art, as are methods of attaching the biosensing elements to the electrode substrate.

The biosensor is then dip coated (or electroplated, or other protocol) in a material such as PEG (of a variety of molecular weights), carboxymethyl cellulose, chitosan, silk protein, or other advantageous mixtures) to achieve a coating that is both fully protective and thin enough to prevent excessive tissue damage during insertion.

The protocol used to apply the coating will depend on the duration of time a coating is required to protect the biosensor (ranging from seconds to days).

Removal of sensor coatings can happen in several ways: 1) physiological conditions such as body temperature and salinity of cerebral spinal fluid may dissolve some types of coatings (which is safe with molecules such as PEG that are used for drug delivery in the body regularly). 2) Reverse electroplating by applying a small current or potential to the coated sensor may disperse the coating from the sensor surface. 3) shearing force during insertion may be used to remove the coating near the surface of the brain, protecting the sensor through the bloodiest area of the surgery, while keep the coating molecules from penetrating neural tissue that will be sensed (which may be important if release of some coating molecules interacts with neural tissue). 4) a protein-based coating (such as silk-I protein polymer) could be removed by endogenous proteases once implanted. Thickness and hydration of coating would determine how long it takes proteases to remove coating layer

In the event that sensors are to be exposed at different time points, a reverse electroplating protocol may be applied to a single sensor at the time. The benefit of this kind of sequential coating release may be prolonged in vivo sensing. If dissolution of coating in physiological environment is the method of coating release, then sensors may have progressively thicker coatings to stagger their exposure to neural tissue.

Patterning of coatings onto microfabricated sensor substrates may be used to more precisely mask/expose certain sensors at desired times.

Additionally, the temporary coating may be impregnated with drugs that have facilitate the recovery from implantation, such as steroids to reduce the immune response or heparin to reduce blood clotting near the surface of the sensor. Through the use of a temporary coatings, these drug molecules would only be around the sensor for the duration of coating dissolution or removal, which is a benefit because the drugs would be present when needed, but not once sensing experiments have begun.

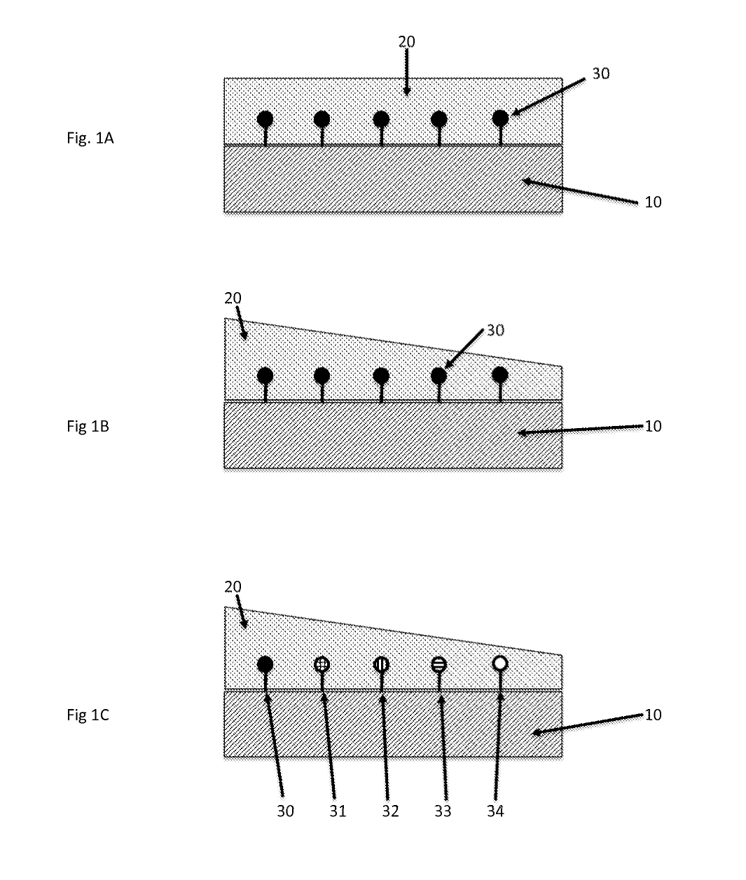

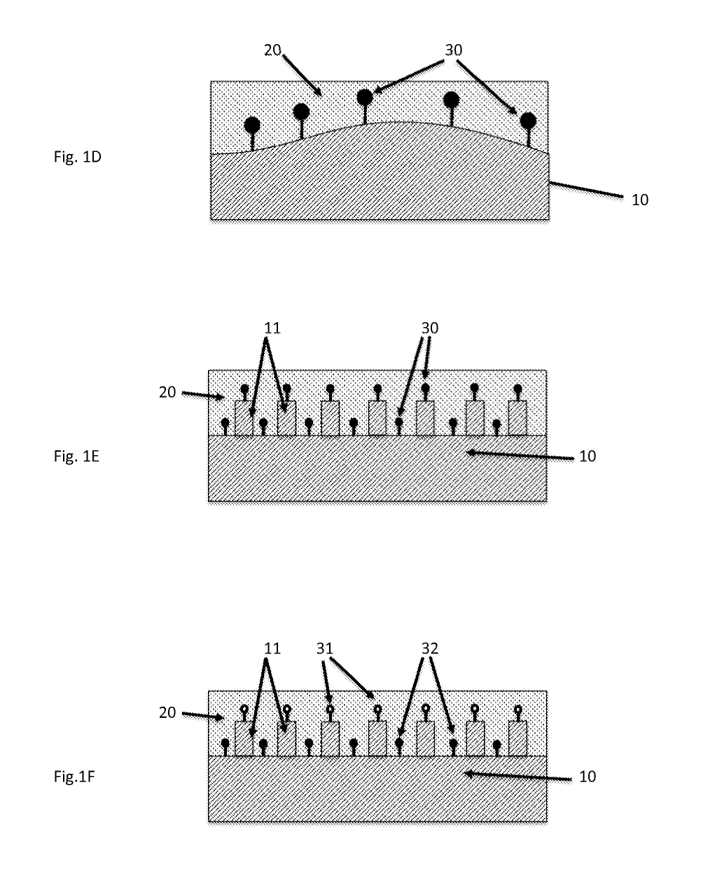

In the embodiment shown in FIG. 1A, an array 10 covered with a coating 20 which covers biosensing elements 30. A modification of this embodiment is shown in FIG. 1B, in which the coating 20 is applied in a manner such that the thickness of the coating 20 is greater at one end of the array 10 than at the other end of the array 10. A single variety of biosensing elements 30 is disposed on the array 10. The variation in the thickness of the coating provides a mechanism whereby, as the coating is eroded, biosensors at one end of the array will be exposed sooner, and biosensors at the other end of the array will be exposed later. FIG. 1C shows a further variation of this embodiment, which employs multiple different biosensing elements 30, 31, 32, 33, 34 disposed on the array 10. In this further variation, as the coating erodes, the sensitivity of the array changes as different types of biosensing elements are exposed.

FIG. 1D shows a schematic of an array 10 where the thickness of the coating 20 varies over the surface of the array because of the underlying topography of the array 10. In this embodiment, biosensing elements 30 that are covered by a thinner layer of the coating 20 will be exposed sooner than biosensing elements 30 that are covered by a thicker layer of the coating 20.

A variation of the embodiment of FIG. 1D is shown in FIGS. 1E and 1F. In the embodiment of FIGS. 1E and 1F, the array 10 is characterized by projections or “pillars” 11. The cross-sectional shape of these pillars may be square, round, or any other shape required. The pillars 11 may be attached to the array 10; alternatively, the array may be manufactured with the pillars as an integral part of the array, either by building up the pillars on the array, or etching away material on the array by, for example, photolithographic or other means.

In the embodiment of FIG. 1E, the biosensing elements 30 bound to the top of the pillars 11 are covered with a thinner layer of the coating 20 than are the biosensing elements 30 which are bound to the array 10 between the pillars 11. As a result, the biosensing elements 30 which are bound to the tops of the pillars 11 will be exposed sooner than the biosensing elements which are bound to the array 10 between the pillars. In a further alternative shown in FIG. 1F, the biosensing elements 31 bound to the tops of the pillars 11 are different (e.g., are sensitive to different target molecules) than are the biosensing elements 30 which are bound to the array 10 between the pillars. In this embodiment, the biosensing elements 31 are exposed sooner than are the biosensing elements 30, because they are covered by a thinner layer of the coating 20.

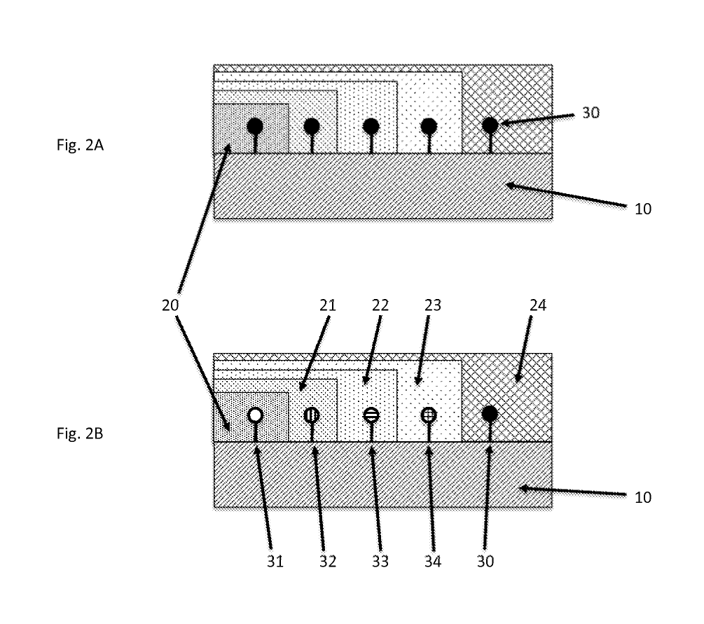

A further alternative embodiment is shown in FIGS. 2A and 2B. In this embodiment the array 10 is covered by multiple coatings 20, 21, 22, 23, 24. Each coating may be selected in such a manner that they can be removed in a controlled sequence, at times desired by the user. In FIG. 2A all of the biosensing elements 30 are the same; in such an array, the different sensing elements are exposed in order to “activate” the array at different desired times. In the variation of this embodiment shown in FIG. 2B, each different coating covers a different biosensing element 30, 31, 32, 33, 34. These elements may be differentially sensitive to a particular target molecule, or they may be sensitive to multiple different targets, or some combination of the two. The embodiment of FIG. 2B allows the user to change the sensitivity of the array by removing the different coatings, thereby exposing a different set of biosensors.

What is claimed is:

1. A functionalized biosensor comprising
one or more biosensing elements
an electrode substrate, and
a coating that covers the biosensing elements.

2. The biosensor of claim 1, wherein the biosensing elements are selected from the group consisting of aptamers, enzymes, and antibodies.

3. The biosensor of claim 1, wherein the electrode substrate is a microwire or microfabricated sensor.

4. The biosensor of claim 1, wherein the coating comprises a material which dissolves under conditions of physiological salinity and temperature.

5. The biosensor of claim 1, wherein the coating comprises a material which is sensitive to endogenous proteases.

6. The biosensor of claim 1, wherein the coating is selected from the group consisting of PEG, carboxymethyl cellulose, and chitosan, silk protein, and mixtures thereof.

7. The biosensor of claim 1, wherein the thickness of the coating is sufficient to protect the biosensors from tissue damage during insertion of the biosensor.

8. The biosensor of claim 1, wherein the coating is electroplated.

9. The biosensor of claim 1, wherein the coating is a dip coating.

10. The biosensor of claim 1, wherein sensor is configured to permit a small current or potential to be applied after implantation in order to disperse the coating by reverse electroplating.

11. The biosensor of claim 1, wherein more than one layer of a coating is applied to the biosensor.

12. The biosensor of claim 1, wherein two or more coatings are applied to the biosensor.

13. The biosensor of claim 12, wherein the coatings are applied in a pattern, such that different sensors are masked with different coatings.

14. The biosensor of claim 1, wherein the coating is impregnated with a drug.

15. The biosensor of claim 14, wherein the drug is a steroid.
